(19) United States
(12) Patent Application Publication
(10) Pub. No.: US 2004/0039062 A1
(43) Pub. Date: Feb. 26, 2004

(54) Urea, ruminally synthesizable mainly to microbial crude protein

(75) Inventor: William P. Moore, Hopewell, VA (US)

Correspondence Address: William P. Moore, P. O. Box 1270, Hopewell, VA, 23860, US

(73) Assignee: Agri-Nutrients Technology Group, Inc.

(21) Appl. No.: 10/227,640

(22) Filed: Aug. 26, 2002

(51) Int. Cl. A61K 31/17; A61K 9/50

(52) U.S. Cl. 514/588

(57) ABSTRACT

A feed urea composition, ruminally synthesizable mainly to microbial crude protein (MCP) in rumen fluid of ruminant animals, the urea particles exhibiting diameters between 0.5 and 1.5 millimeters, coatingly covered with a non-rumen-degradable semipermeable resin membrane exhibiting a thickness of between 3 and 15 microns and a permeability which allows between 20 and 70 percent of the urea to dissolve in water at 39° C. in 10 hours. Effective semipermeable resin membranes are thermoplastic and thermosetting resins including polyolefins and polyurethanes, and the permeabilities of the membranes may be controlled by the addition of one, or more, additives, including triethanolamine, polyalkylene glycols, and clays.

### BACKGROUND OF THE INVENTION

1. Field of the Invention

The present invention relates to animal nutrition and to the use of non protein nitrogen (NPN) as an economical source of protein replacement in the feed rations of ruminant animals. More particularly, it relates to a composition of feed urea particles, coated with a semipermeable membrane of resin, which are converted mainly to ruminally synthesized microbial crude protein (MCP) in the rumen of ruminant animals. The composition is effective when it releases roughly half of its urea content into water in ten hours, using a water solubility test. The conversion of urea to MCP in the rumen is related to water dissolution rate of the coated urea. The optimum conversion occurs within a limited range of water dissolution rates.

2. Description of Prior Art

Urea, and to a lesser degree ammonium salts, have been successfully fed to ruminant animals as replacements for limited amounts of protein. These materials are usually called non protein nitrogen (NPN). NPN products are largely fed to dairy and feedlot cattle. Economic benefits have been shown from the limited amounts of NPN which may be safely fed in lieu of natural proteins. Prior art work has been focused on limiting ammonia concentrations generated in rumen fluids from urea. The prior art has provided no teachings on effective formation of ruminally synthesized microbial crude protein (MCP) and its optimization.

MCP is a term used by ruminant researchers. It is defined as protein and amino acids synthesized by microbial actions from various nitrogen and energy sources in the rumen for absorption in the post ruminal digestive tract.

When urea, or other NPN source, is fed to ruminants, it is initially quickly broken down to ammonia and carbon dioxide by urease found in feed rations and also produced by certain microorganisms. Paralleling this catabolism of urea in the rumen, carbohydrates are degraded by other ruminant microorganisms to form volatile fatty acids and keto acids. The ammonia liberated by urease in the rumen combines with available keto acids to form amino acids, which are to a large extent, in turn, incorporated into microbial protein. Throughout the digestion of a ruminant animal, microorganisms are passed from the rumen to the more distal digestive organs. In the abomasum and small intestines these microbes are hydrolyzed and digested to such a degree that the microbial protein is broken down to free amino acids which can then be absorbed by the host animal.

It has been known for a long time to use urea as a replacement for small amounts of natural protein in the feed of ruminant animals, but the amount of urea used has been severely limited to preclude ammonia toxicity in the animal. No prior art worker has shown a high degree of ruminal synthesis of crude microbial protein (MCP) from urea via ammonia in the rumen.

There are several technologies in the art where semipermeable membranes have been used to control the release of nitrogen fertilizer compounds in water and soil. A semipermeable membrane is defined herein as a microporous structure which acts as a highly efficient filter in the range of molecular dimensions, allowing passage of ions, water, other solvents, and very small molecules, but being almost impervious to macromolecules and colloidal particles. The semipermeable membranes of interest in the instant invention are thick enough to allow the slow diffusion of soluble molecules and are about three to fifteen microns thick.

Emanuele, S. M. et al in U.S. Pat. No. 6,231,895 disclose a ruminant feedstock with a content of non protein nitrogen supplement which yields a safe controlled-release generation of ammonia under rumen incubation conditions. The non protein nitrogen comprises urea particles encapsulated with a rumen-degradable polymeric coating. No teaching or suggestion was provided for the use of thin non-rumen-degradable semipermeable membrane coatings to accurately release the soluble NPN nitrogen compounds while the coating shells remain intact. Although no specific amounts of coatings are specified, the examples provided substantial coatings amounting to more than 6 percent of the nitrogen supplement.

In my copending U.S. patent application Ser. No. 10/128,279 a concentrated controlled release NPN ruminant feed supplement composition is disclosed which releases nitrogen in rumen fluids at about the same rate that nitrogen metabolizes therein. A non-rumen-degradable semipermeable membrane coatingly covers the urea particles to allow substantially complete diffusion of the urea through the membrane in 6 to 24 hours. No teachings were made of conversions of a high degree of the urea nitrogen to MCP or of a method of analyses to determine the amount of nitrogen convertible to MCP.

In summary, none of the prior art teaches, or suggests that a coated urea composition may be prepared so that the urea nitrogen fed to a ruminant animal is ruminally synthesized mainly to MCP and that ruminally synthesized MCP from the urea composition may be optimized by maintaining the rate of water solubility of the composition within a narrow range in a water solubility test.

### OBJECTS OF THE INVENTION

It is therefore an object of this invention to provide a coated urea composition which, when fed to ruminant animals, is ruminally synthesized mainly to MCP.

It is a further object of this invention to provide the limits within which the composition must be held so that the urea is ruminally synthesized mainly to MCP.

It is a further object of this invention to provide variations of the composition within limits to make the composition effective in different ruminant feeding rations.

It is a further object of this invention to provide a test method to assure that the composition is effective and within the required limits.

It is a further object of this invention to provide an economical method of preparing the effective composition.

### SUMMARY OF THE INVENTION

I have now discovered a coated urea composition which, when fed to ruminant animals, is ruminally synthesized mainly to microbial crude protein. The urea is coatingly covered with a non-rumen-degradable semipermeable resin membrane. The permeability of the semipermeable membrane must be in the narrow range of the instant composition to provide ruminal synthesis of the urea via ammonia mainly to microbial crude protein (MCP). Adjustments of the thickness and the inherent permeability of the coatings can be made within the narrow composition range of the invention which can be used to optimize the permeability of the membrane for any ordinary ruminant feed ration. Surprisingly, a simple water solubility test may be used to assure that ruminal synthesis of MCP from urea via ammonia is optimized. To assure mainly synthesis of MCP, it is necessary that between 20 and 70 percent of the urea in the composition be dissolved in a water solubility test at 39° C. in 10 hours. Variations of inherent permeability of the semipermeable resin membranes may be achieved by use of resins with different properties, or by admixing in the resin membranes permeability-changing materials.

### DETAILED DESCRIPTION OF THE INVENTION

This invention is directed to a feed urea composition which is ruminally synthesizable mainly to microbial crude protein (MCP) in ruminant animals consuming the composition and ordinary feed rations. The new composition contains urea particles amounting to more than 85 percent of the total composition. Compositions containing lesser amounts of urea may be prepared but leave undesirably large residues of unused urea and coatings and are uneconomical.

A non-rumen-degradable semipermeable resin membrane coatingly covers the urea particles, the membrane thickness amounting to between 3 and 15 microns. Thinner coatings release urea rapid enough to cause undesirably high ammonia concentrations in the rumen which create animal toxicity concerns and poor ruminal synthesis to MCP. Thicker concentrations release urea too slow for efficient synthesis of MCP during its residence time in the rumen.

The membrane exhibits a permeability which allows between 20 and 70 percent of the urea to be dissolved in a water solubility test in 10 hours at 39° C. The water solubility test is an accurate method to determine whether the coated urea will be ruminally synthesized mainly to MCP by ruminant animals consuming the composition.

The urea of the instant composition is ruminally synthesized via ammonia mainly to MCP when the composition provides a total nitrogen concentration in the rumen fluid derived from urea amounting to between 0.02 and 0.20 percent. Lower concentrations provide inadequate amounts of MCP and higher concentrations create ammonia toxicity concerns. Preferred composition performance is obtained when the total nitrogen concentrations from urea amounts to between 0.08 and 0.20 percent.

It is important that the instant composition comprise urea particles exhibiting diameters between 0.50 and 1.50 millimeters. Larger diameters are ineffective because they are sometimes chewed by the ruminant animals destroying the coatings, and sometimes sink to the bottom of the rumen and are not effectively synthesized. Smaller particles are difficult to coat economically although they may be used effectively when coated with the requisite thickness of resin membrane.

A variety of semipermeable thermoplastic resin membranes may be used in the instant composition including polyolefins, nylons, and low molecular weight polyurethane polymers. Thermosetting resin membranes including alkyds, amino resins, polyesters, and cross-linked polyurethane resins may also be effectively used.

The thickness of the resin membranes preferably amounts to between 5 and 10 microns. The thickness rather than the weight percent is the measurement controlling coating efficacy because the weight percent coating on a particle for a given thickness varies widely depending upon the diameter of the coated particle.

In the preferred composition, the permeability of the resin membrane is controlled so that between 20 and 55 percent of the urea is dissolved in a water solubility test at 39° C. in 10 hours.

A most preferred composition of the instant invention exhibits a permeability of the resin membrane controlled so that between 30 and 80 percent of the urea is ruminally synthesized to MCP.

The instant composition exhibits a distinct advantage over prior art urea forms in that it may be used in a wide variety of feed rations. Substantial amounts of the composition may be used effectively in feed rations for dairy cows, beef cattle, and feed lot cattle. It may also be used in range cattle feed supplements; in feed rations containing high energy, low energy and high fiber contents; and in feed rations utilizing high non protein nitrogen contents.

The composition of the instant invention may be varied within the prescribed limits to fit feed ration requirements where the non-rumen-degradable semipermeable membrane resin contains one or more additives to control the inherent permeability of the membrane.

Additives effective in controlling the permeability of the membrane include alkanolamines, alkylene glycols, rubber latexes, waxes, polyalkylene glycols, clays and diatomaceous earth.

In a preferred feed urea composition, wherein the urea is ruminally synthesized to MCP with an efficiency of between 40 and 80 percent in cattle receiving the composition and ordinary feed rations, spheroid urea exhibiting diameters between 0.75 and 1.50 millimeters is used. Particles in this size range are not chewed by the cattle, do not sink and stay at the bottom of the rumen, and exhibit effective synthesis to MCP in about the usual retention time in the rumen.

In a preferred composition, a non-rumen-degradable semipermeable polyurethane resin membrane coatingly covers the urea particles, and the membrane exhibits a thickness between 5 and 10 microns and a permeability controlled so that between 20 and 55 percent of the urea is dissolved in a water solubility test at 39° C. in 10 hours.

In a preferred composition triethanolamine permeability controlling additive is homogeneously admixed in the resin membrane amounting to between 1 and 10 percent of the resin membrane.

### MODE OF OPERATION OF THE INVENTION

Having described the basic concepts of the instant invention, reference is now made to the following examples which are provided to illustrate the composition of this invention and its utility.

### EXAMPLE 1

This example demonstrates a preferred composition of this invention including its preparation and control analysis.

A stainless steel 8 cubic foot capacity Gemco batch double cone vacuum dryer, jacketed for steam heating and water cooling, and equipped with a liquid spray bar running horizontally along the center of the rotation of the dryer, was charged with 200 pounds of urea granules exhibiting diameters between 0.6 and 1.4 millimeters at 93° C.

Rotation was started on the double cone dryer at a rate of 20 rpm. A liquid resin mixture comprising 92 percent Witcobond W-170, a fully cured polyurethane latex containing 35 percent resin, no solvent, and 65 percent water was selected. The liquid resin mixture selected also contained 4 percent Cymel 373 a metholated methoxy melamine crosslinking agent, and 4 percent diatomaceous earth permeability control agent.

A vacuum system connected to the double cone dryer was started and the pressure in the double cone dryer and the urea granules therein was reduced to 40 millimeters mercury. The liquid resin mixture amounting to 2.5 pounds (containing 1.0 pound dry matter) was introduced through the spray bar through a 2 minute period. Rotation was continued in the double cone dryer for 5 minutes at a temperature between 79 and 92° C. to allow the resin to dry this first dose of resin into a layer of non-rumen-degradable semipermeable membrane.

The introduction and drying of the liquid resin doses was repeated until the thickness of the membrane coatingly covering the urea was calculated to be 8 microns.

The coated urea was retained under vacuum for 15 minutes at 83° C. after the 8 micron coating level was reached, and then cooled to 32° C. and discharged at atmospheric pressure.

Water solubility rate of the coated urea was determined by placing a 5 gram sample in 200 grams of distilled water at 39° C. and measuring the amount of urea dissolved after 10 hours in the water. The mixture was swirled briefly only when the sample was added and at the end of 10 hours. The percent of the urea dissolved in 10 hours was measured to be 45 by weight.

The coating was analyzed to be 5.6 percent by weight of the coated granule.

### EXAMPLE 2

This example demonstrates that the feed urea composition of this invention is ruminally synthesizable mainly to microbial crude protein (MCP).

A series of urea conversion in-vitro tests were arranged using the following materials:

(1) Fresh rumen fluid withdrawn via a pump through a strainer from a heifer receiving no feed for the prior two days.

(2) Simulated Saliva composition of E. I. McGougal in Biochemical Journal, Vol. 43, page 106.

(3) Cellulose energy source, Whatman Chromedia Microgranular Cellulose, free from other nutrient compounds.

(4) The feed urea product of Example 1.

The in-vitro tests were run with each sample in a 250 ml Erlenmeyer flask receiving the ingredients listed as follows:

| Materials | Wt, grams |
|---|---|
| Simulated Saliva | 25 |
| Fresh Rumen Fluid | 175 |
| Cellulose Energy | 1 |
| Example 1 Product | 0.4065 |

The test flasks were placed in a constant temperature bath operating at 39° C. Carbon dioxide was used to displace the air in the flasks. Samples amounting to 10 grams were withdrawn on an accurate time schedule with the air in the sample container replaced by carbon dioxide. The samples were immediately treated with 1 percent mercuric chloride to terminate microbial activity in the samples. The samples were analyzed for total nitrogen, ammonia nitrogen, and MAC by difference.

The results obtained from the analyses after 8 hours of ruminal synthesis of urea to MCP are tabulated as follows:

| | | | | Conversion |
|---|---|---|---|---|
| | Nitrogen Concentrations, Wt % | | | of Urea to |
| | Total from | | | MCP |
| Nitrogen Source | Urea | NH₃-N | MCP | % |
| Example 1 Product | 0.107 | 0.039 | 0.068 | 63.5 |

The urea was ruminally synthesized mainly to microbial crude protein in rumen fluid in 8 hours, which is about the normal retention time for materials passing through the rumen of ruminant animals.

### EXAMPLE 3

This example demonstrates variations by several different means of the permeability of the non-rumen-degradable semipermeable resin membrane causing changes in the water solubility rates of the urea, and how the synthesis of MCP from the urea relates to the water solubility rates. The example also shows comparative data taken with uncoated urea, urea coated with rumen degradable resins, and with soybean meal.

Three preparations of the composition of this invention were made using the apparatus and the general operation and ingredients of Example 1. Variations were made in the thickness of the coating in one product and the inherent permeability of the membrane was varied in another product by inclusion of clay as additive. A similar preparation was made in which a rumen-degradable coating was used on larger urea granules. Comparative in-vitro measurements with the apparatus and procedures of Example 2 were made with each of the varied feed urea products and with uncoated feed urea and with soybean meal. The results of the nearly isonitrogenous tests are tabulated in the following table.

| | | | | | | | |
|---|---|---|---|---|---|---|---|
| Test No. | 1 | 2 | 3 | 4 | 5 | 6 | 7 |
| Diameter of Urea, mm | 0.83 | 0.83 | 0.83 | 2.36 | 0.83 | 0.83 | Soy |
| | | | | | | | meal |
| Type of Coating | A | A | A | B | A | — | — |
| Coating Additive | — | — | — | — | C | — | — |
| Amount of Additive, % | — | — | — | — | 7.5 | — | — |
| Thickness of Coating, mm | 5.5 | 7.8 | 10.2 | 11.0 | 8.2 | 0 | — |
| Urea Solubility, 39° C. (10 hours) | 70 | 44 | 28 | 74 | 55 | 100 | — |
| Total N Content, Wt % (8 hours) | 0.11 | 0.11 | 0.11 | 0.08 | 0.09 | 0.09 | 0.09 |
| NH₃ Conc., Wt % (8 hours) | 0.069 | 0.039 | 0.025 | 0.074 | 0.048 | 0.087 | 0.010 |
| Urea Synthesized to MCP, % | 35.9 | 63.4 | 76.4 | 13.3 | 47.0 | 3.6 | 88.8 |

Notes:
A: rumen-non-degradable thermosetting crosslinked polyurethane
B: rumen-degradable thermoplastic polyurethane + 6 percent vegetable oil
C: A + 5 percent diatomaceous earth clay, through 150 mesh screen

The results on tests 4 and 6 show that urea either uncoated or coated with rumen-degradable resin exhibited high urea water solubilities at 39° C. and correspondingly low synthesis efficiencies to MCP. The release of nitrogen and formation of ammonia therefrom started out slowly by osmosis for the first few hours in rumen juices and then rapidly increased as the rumen degraded the coatings rapidly so that after 8 hours in the rumen the MCP formation was not much better than that obtained with uncoated feed urea.

The ureas coated with non-rumen-degradable semipermeable membranes increased ruminally synthesized MCP as the water solubility test results decreased. Where the permeability of the semipermeable membrane was increased by adding diatomaceous earth, the MCP synthesis was about the same as that obtained with the same water solubility obtained with a thinner coating containing no clay. The ruminally synthesized MCP for the high quality soybean meal was 88.8 percent which was slightly higher than the 76.4 percent obtained with Test 1 using the instant feed urea composition.

I claim:

1. A feed urea composition, ruminally synthesizable mainly to microbial crude protein (MCP) in ruminant animals consuming the composition and ordinary feed rations, the composition comprising:
(a) urea particles amounting to more than 85 percent of the composition; and,
(b) a non-rumen-degradable semipermeable resin membrane coatingly covering the urea particles, the membrane exhibiting a thickness amounting to between 3 and 15 microns and a membrane permeability which allows between 20 and 70 percent of the urea to be dissolved in water in 10 hours at 39° C.

2. The composition of claim 1 wherein the urea ruminally synthesized mainly to MCP provides a total nitrogen concentration derived from urea in the rumen fluid amounting to between 0.02 and 0.20 percent.

3. The composition of claim 1 wherein the urea ruminally synthesized mainly to MCP provides a total nitrogen concentration derived from urea in the rumen fluid amounting to between 0.08 and 0.20 percent.

4. The composition of claim 1 wherein the urea particles exhibit diameters between 0.50 and 1.50 millimeters.

5. The composition of claim 1 wherein the semipermeable resin membrane comprises a thermoplastic resin selected from the group consisting of polyolefins, nylons, and low molecular weight polyurethane polymers.

6. The composition of claim 1 wherein the semipermeable resin membrane comprises a thermosetting resin selected from the group consisting of alkyds, amino resins, polyesters, and crosslinked polyurethanes.

7. The composition of claim 1 wherein the resin membrane thickness amounts to between 5 and 10 microns.

8. The composition of claim 1 wherein the permeability of the resin membrane is controlled so that between 20 and 55 percent of the urea is dissolved in water in a water solubility test at 39° C. in 10 hours.

9. The composition of claim 1 wherein the permeability of the resin membrane is controlled so that between 30 and 80 percent of the urea is ruminally synthesizable to MCP.

10. The composition of claim 1 wherein the ordinary feed rations are selected from the group of feed rations consisting of dairy cow, beef cattle, feed lot cattle, range cattle feed supplements, high energy, low energy high fiber, and high non-protein nitrogen.

11. The composition of claim 1 wherein the non-rumen-degradable semipermeable membrane resin contains one or more additives to control the permeability of the membrane.

12. The composition of claim 11 wherein the permeability controlling additive is selected from the group consisting of alkanolamines, alkylene glycols, rubber latexes, waxes, polyalkylene glycols, clays, diatomaceous earth.

13. A feed urea composition ruminally synthesizable to MCP with an efficiency between 40 and 80 percent in cattle receiving the composition and ordinary feed rations, the composition comprising:
(a) spheroid urea particles exhibiting diameters between 0.75 and 1.50 millimeters;
(b) a non-rumen-degradable semipermeable polyurethane resin membrane coatingly covering the urea particles and exhibiting a thickness of between 5 and 10 microns and permeability controlled so that between 20 and 55 percent of the urea is dissolved in a water solubility test at 39° C. in 10 hours; and,
(c) triethanolamine permeability controlling additive homogeneously admixed in the resin membrane amounting to between 1 and 10 percent thereof.
